(19) United States
(12) Patent Application Publication
(10) Pub. No.: US 2012/0041300 A1
(43) Pub. Date: Feb. 16, 2012

(54) METHOD FOR CONTRAST AGENT-FREE MAGNETIC RESONANCE COLONOGRAPHY

(76) Inventors: Walter Maerzendorfer, Erlangen (DE); Martin Requardt, Nuernberg (DE); Sebastian Schmidt, Weisendorf (DE)

(21) Appl. No.: 13/210,577

(22) Filed: Aug. 16, 2011

(30) Foreign Application Priority Data
Aug. 16, 2010 (DE) 10 2010 039 348.7

(51) Int. Cl. A61B 5/055

(52) U.S. Cl. 600/420

(57) ABSTRACT

In a method for contrast agent-free magnetic resonance colonography, blood that flows into the bowel tissue is prepared automatically by radio-frequency pulses. Magnetic resonance data of the bowel are acquired while the prepared blood is located in the bowel, and a magnetic resonance image of the bowel is created from the magnetic resonance data. Bowel tissue regions and regions with bowel content are differentiated automatically on the basis of an increased contrast (caused by the preparation) between perfused tissue regions and regions of the gut lumen that are not perfused.

### BACKGROUND OF THE INVENTION

1. Field of the Invention

The present invention concerns a method for a contrast agent-free magnetic resonance colonography as well as a magnetic resonance system for a magnetic resonance imaging in a contrast agent-free colonography.

2. Description of the Prior Art

Magnetic resonance colonography, also known as virtual colonoscopy, is a method in which magnetic resonance data of the bowel of a patient are acquired with the use of a magnetic resonance tomography apparatus and the determined data are subsequently converted into a presentation form that corresponds to a conventional colonoscopy. This examination thus can replace a conventional colonoscopy implemented by endoscopy in many cases.

Since the data set exists as a volume data set—which is different than in conventional colonoscopy, which generates only 2D images—other presentation forms can also be generated that go beyond the possibilities of endoscopic colonoscopy. For example, an unfolding of the bowel and a 2D presentation of the entire bowel surface as described in U.S. 2009/0225077 A1; a panorama presentation as in U.S. Pat. No. 7,609,910 B2; a marking of already viewed regions as described in U.S. 2008/0175459 A1.

In data acquisitions for magnetic resonance colonography, it is often difficult to achieve a good demarcation between the bowel wall and the bowel content. In order to achieve this necessary good demarcation between bowel wall and bowel content, methods known as “dark lumen” methods have been implemented, for example. In this method the bowel is filled with gas (air, for example) and a contrast agent (for example a gadolinium chelate) is additionally administered intravenously. This contrast agent is required in order to improve the contrast between the well-perfused bowel wall and the bowel content and to thereby enable a segmentation and better diagnosis. In particular, the contrasting facilitates the differentiation of bowel content (for example stool residues) and perfused polyps that protrude into the gut lumen.

For medical reasons this intravenous contrast agent administration is not desirable because it entails risk, for example contrast agent allergies or kidney diseases. In particular, an intravenous contrast agent administration is undesirable when healthy people are examined within the scope of a screening. Magnetic resonance contrast agents for such specific purposes are not explicitly permitted by the regulatory agencies in various countries, so additional legal risks exist. The contrast agent administration additionally requires an intravenous injection with appropriate injection speed, which reduces the acceptance with the patients.

In addition, techniques known as “bright lumen” methods are available, in which an intravenous injection can be avoided. In this method the bowel is filled with a contrast-providing fluid. However, due to poorer clinical results such methods have not found a wide acceptance.

An additional problem of modern magnetic resonance colonography is that a complicated bowel evacuation is required in order to safely eliminate stool residues in the bowel. However, the acceptance with the patients is thereby reduced because the method is thereby not significantly more comfortable than a conventional endoscopic colonoscopy.

### SUMMARY OF THE INVENTION

An object of the present invention is to provide a magnetic resonance colonography technique that functions without an intravenous contrast agent injection and that provides a good demarcation between bowel wall and bowel contents, even given an incomplete bowel evacuation.

Within the scope of the present invention, a method is provided for a contrast agent-free magnetic resonance colonography, wherein blood flowing into the bowel tissue is prepared by radiation of radio-frequency pulses, and magnetic resonance data are acquired while the prepared blood is located in the bowel. A magnetic resonance image of the bowel is created from the acquired magnetic resonance data. Bowel tissue regions and regions with bowel contents are differentiated automatically on the basis of an increased contrast (caused by the preparation of the blood flowing into the bowel) between perfused tissue regions and regions within the bowel that are not perfused (which thus represent a bowel content).

By the preparation of incoming blood—known as arterial spin marking—perfused tissue regions of the bowel, and in particular the bowel wall, can be identified reliably based on the increased contrast. Since polyps that are present in the gut lumen are also possibly perfused, a reliable differentiation between polyps and bowel content is possible. Moreover, a statement regarding perfusion of the polyps can be made and serve as a measure for the risk of a malignancy. Due to the improved separation of bowel content and perfused bowel wall and possibly perfused polyps, this method enables examinations even without a complete bowel cleaning. Moreover, no administration of contrast agent—in particular no intravenous contrast agent administration—is required, so the acceptance with the patient can be increased.

According to one embodiment of the method, further magnetic resonance data are additionally acquired while unprepared blood is located in the bowel tissue. An additional magnetic resonance image is created automatically from these additional magnetic resonance data. Perfused bowel tissue regions and regions with bowel contents are differentiated automatically on the basis of the magnetic resonance image which was created with the prepared blood and the additional magnetic resonance image which was created without the prepared blood. For example, the two magnetic resonance images can be compared automatically, and regions that are essentially the same in all magnetic resonance images can be associated with the bowel contents. Alternatively or additionally, a difference image between the magnetic resonance image with prepared blood and the additional magnetic resonance image without prepared blood can also be determined automatically, and regions of the bowel content thus can be masked out since the bowel content is not perfused and therefore shows essentially the same signal values in the two images. An easier differentiation of bowel tissue and regions with bowel content is thereby possible, so three-dimensional representations can be reliably created, for example.

In a further embodiment, the aforementioned magnetic resonance data—i.e. the magnetic resonance data while prepared blood is located in the bowel tissue and the magnetic resonance data while unprepared blood is located in the bowel tissue—are acquired with a low resolution, meaning that the corresponding magnetic resonance images have a low resolution. A low resolution in this context means, for example, a resolution reduced by a factor of two to four relative to a high resolution (which, for example, can be the maximum resolution of the magnetic resonance system and which is advantageously used by a physician for a diagnostic evaluation). In addition to these two magnetic resonance images with low resolution, an additional magnetic resonance image with high resolution is created from corresponding, additionally acquired magnetic resonance data. The differentiation between bowel tissue and bowel content is implemented using the magnetic resonance images with low resolution. Regions that are associated with the bowel content are then automatically presented as empty regions in the magnetic resonance image with high resolution. The two magnetic resonance images with low resolution are subtracted from one another, for example, and thus a mask is formed which masks the bowel content. The difference image is subsequently applied to the diagnostic magnetic resonance image with high resolution in order to automatically remove the bowel content in said image with high resolution. This mask can additionally be improved with methods of image processing, for example in that algorithms for edge detection, smoothing, etc. are applied.

In a further embodiment, additional oral substances can be administered that affect the contrast of the bowel content. For example, insoluble barium compounds (barium sulfate, for example) can be used in order to suppress the T1 signal from the gut lumen. Alternatively, iron compounds (iron oxides, for example) can be used. The differentiation of bowel tissue regions and regions with stomach content can thereby be implemented automatically and reliably based on the increased contrast. The additional substances that are administered orally are generally more agreeable than intravenously administered contrast agents, such that the acceptance of the use of the oral substances with the patient is markedly better.

According to a further embodiment, the preparation—i.e. the spin marking—of blood flowing into bowel tissue is implemented transversely in an excitation slice above the diaphragm. Under consideration of a flow speed of the blood of two to four meters per second and a distance of around 20 cm between the excitation slice above the diaphragm and the bowel, a relatively reliable saturation of the spin-marked, incoming blood results for radio-frequency pulses of 2-5 milliseconds. The excitation slice can extend cranially up to the aortic arch.

According to a further embodiment, the blood flowing into the bowel tissue is prepared in an excitation region which comprises the descending aorta at the level of the passage through the diaphragm. This selective excitation of a relatively small volume can be implemented, for example, with the use of a multichannel transmission system, known as a parallel transmit system. it can thereby be ensured that only blood flowing through the aorta is marked.

According to a further embodiment, individual branches of the mesenteric artery are selectively excited. A relatively small blood volume can thereby be prepared directly in proximity to the region of interest. The vessel relationships in the region of interest can thereby be examined more precisely at the same time, in particular the supply areas of the superior and inferior mesenteric arteries, which can be important for a subsequent operation planning.

According to a further embodiment, in a first automatic preparation of blood flowing into the bowel tissue a sagittal slice along the aorta is excited with the aid of radio-frequency pulses. First magnetic resonance data of the bowel are acquired while the prepared blood is located in the bowel tissue. Given a second automatic preparation of blood flowing into the bowel tissue with the aid of radio-frequency pulses, a coronal slice along the aorta is excited. Second magnetic resonance data of the bowel are acquired while this prepared blood is located in the bowel tissue. The magnetic resonance image of the bowel can then be created from the first and second magnetic resonance data, wherein the shadow of the marking slices can be compensated via combination of the first and second magnetic resonance data. A more robust saturation of the marking of the blood flowing into the bowel tissue can thereby be achieved.

Within the scope of the present invention, a magnetic resonance system is furthermore provided for magnetic resonance imaging in contrast agent-free magnetic resonance colonography. The magnetic resonance system includes a basic field magnet, a gradient field system, a radio-frequency antenna and a control device. The control device serves to activate the gradient field system and the radio-frequency antenna, to receive measurement signals acquired by the radio-frequency antenna acquired by the radio-frequency antenna, to evaluate the measurement signals and to create magnetic resonance images. The magnetic resonance system is designed to prepare blood which flows into bowel tissue with the aid of radio-frequency pulses, and to acquire magnetic resonance data while the prepared blood is located in the bowel tissue. The magnetic resonance system creates a corresponding magnetic resonance image on the basis of the magnetic resonance data. In the magnetic resonance image the magnetic resonance system automatically differentiates bowel tissue regions and regions with bowel content. This differentiation is based on an increased contrast (caused by the preparation) between perfused tissue regions of the bowel and non-perfused tissue regions in the region of the bowel content. The magnetic resonance system is therefore suitable for implementation of the previously described method and therefore significantly comprises the advantages of the previously described method according to the invention, such that a repetition of the description of the advantages is omitted here.

The magnetic resonance data can be a volume data set, such that three-dimensional magnetic resonance images can be created therefrom. Moreover, other presentation shapes can also be generated from the volume data set, for example a presentation of an unfolded bowel and a two-dimensional presentation of the complete bowel surface, a panorama presentation or a presentation with a marking of already considered regions.

Furthermore, the present invention encompasses a non-transitory, computer-readable storage medium encoded with a computer program (programming instructions) that can be loaded into a memory of a programmable controller or a computer of a magnetic resonance system. All or various embodiments of the method according to the invention that are described above can be implemented with this computer program when executed in the controller or control device of the magnetic resonance system. The computer program may require libraries and auxiliary functions, for example, in order to realize the corresponding embodiments of the method. The software can be source code (for example C++ or Java) that must still be compiled (translated) and linked or that only must be interpreted, or can be an executable software code that need only be loaded into the corresponding computer for execution.

The computer-readable storage medium can be for example, a DVD, a CD, a magnetic tape or a USB stick on which electronically readable control information, in particular software is stored. All embodiments according to the invention of the method described above can be implemented when this control information (software) is read from the data medium and stored in a controller or, respectively, control unit of a magnetic resonance system.

### BRIEF DESCRIPTION OF THE DRAWINGS

FIG. 1 schematically depicts a magnetic resonance system according to the invention.

FIG. 2 shows a program workflow diagram of a method according to the invention.

FIG. 3 shows an attitude of an excitation slice according to one embodiment of the present invention.

FIG. 4 shows an attitude of an excitation slice according to one embodiment of the present invention.

### DESCRIPTION OF THE PREFERRED EMBODIMENTS

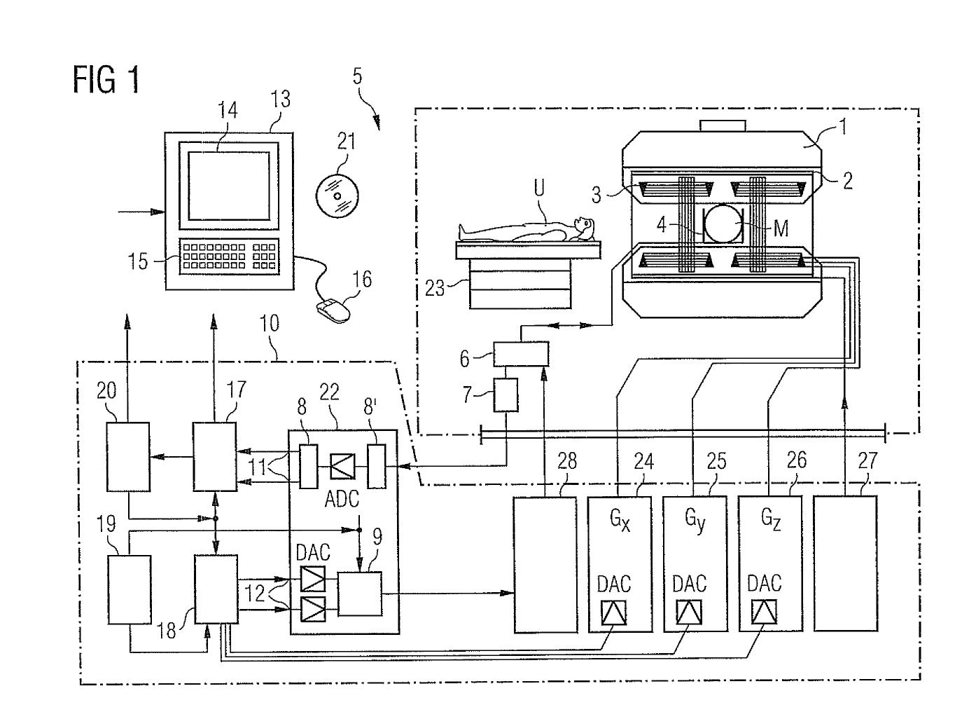

FIG. 1 shows a schematic depiction of a magnetic resonance system 5, known as a magnetic resonance imaging or magnetic resonance tomography apparatus. A basic field magnet 1 generates a temporally constant, strong magnetic field for polarization or, respectively, alignment of the nuclear spins in an examination region of an examination subject, for example of a part (for example the bowel) of a human body U that is to be examined, which body lies on a table 23 and is moved into the magnetic resonance system 5. The high homogeneity of the basic magnetic field that is required for the nuclear magnetic resonance measurement is defined in a typically spherical measurement volume M into which the parts of the human body that are to be examined are introduced. Shim plates made of ferromagnetic material are attached at suitable points to assist the homogeneity requirements, and in particular to eliminate temporally invariable influences. Temporally variable influences are eliminated by shim coils 2 and a suitable activation 27 for the shim coils 2.

A cylindrical gradient coil system 3 having three sub-windings is used in the basic field magnet 1. Each sub-winding is supplied with current by a corresponding amplifier 24-26 to generate a linear gradient field in the respective direction of the Cartesian coordinate system. The first sub-winding of the gradient field system 3 generates a gradient Gₓ in the x-direction; the second sub-winding generates a gradient G_{y} in the y-direction; and the third sub-winding generates a gradient G_{z} in the z-direction. The amplifiers 24-26 each include a digital/analog converter (DAC), activated by a sequence controller 18 for time-accurate generation of gradient pulses.

At least one radio-frequency antenna 4 is located within the gradient field system 3. The radio-frequency antenna 4 converts the radio-frequency pulses emitted by a radio-frequency power amplifier into an alternating magnetic field for excitation of the nuclei and alignment of the nuclear spins of the subject to be examined or of the region of the subject that is to be examined. The radio-frequency antenna 4 includes one or more RF transmission coils and multiple RF reception coils in the form of an annular, linear or matrix-like arrangement, for example. The alternating field emanating from the precessing nuclear spins—i.e. normally the nuclear spin echo signals caused by a pulse sequence made up of one or more radio-frequency pulses and one or more gradient pulses—is also converted by the RF reception coils into a voltage (measurement signal) that is supplied via an amplifier 7 to a radio-frequency reception channel 8, 8′ of a radio-frequency system 22. The radio-frequency system 22 furthermore comprises a transmission channel 9 or multiple transmission channels (not shown) in which the radio-frequency pulses are generated for the excitation of the nuclear magnetic resonance. The respective radio-frequency pulses are digitally represented in the sequence controller as a series of complex numbers based on a pulse sequence predetermined by the system computer 20. This number sequence is supplied as a real part and imaginary part to a digital/analog converter (DAC) in the radio-frequency system via a respective input 12 and from said digital/analog converter (DAC) to the transmission channel 9. In the transmission channel 9 the pulse sequences are modulated on a radio-frequency carrier signal whose base frequency corresponds to the resonance frequency of the nuclear spins in the measurement volume. The modulated pulse sequences of the RF transmission coils are supplied to the radio-frequency antenna 4 via an amplifier 28.

Switching between transmission operation and reception operation takes place via a transmission/reception diplexer 6. The RF transmission coil of the radio-frequency antenna 4 radiates the radio-frequency pulses for excitation of the nuclear spins into the measurement volume M and scans resulting echo signals via the RF reception coils. The acquired nuclear magnetic resonance signals are phase-sensitively demodulated on an intermediate frequency in a first demodulator 8′ of the reception channel of the radio-frequency system 22 and digitized in an analog/digital converter (ADC). This signal is further demodulated on a frequency of zero. The demodulation on a frequency of zero and the separation into real part and imaginary part occurs in a second demodulator 8 after the digitization in the digital domain, which demodulator 8 outputs the demodulated data to an image computer 17 via outputs 11. An MR image is reconstructed by the image computer 17 from the measurement data acquired in such a manner. The administration of the measurement data, the image data and the control programs takes place via the system computer 20. Based on a specification with control programs, the sequence controller 18 monitors the generation of the respective desired pulse sequences and the corresponding scanning of k-space. In particular, the sequence controller 18 thereby controls the time-accurate switching of the gradients, the emission of the radio-frequency pulses with defined phase amplitude and the reception of the nuclear magnetic resonance signals. The time base (clock signals) for the radio-frequency system 22 and the sequence controller 18 is provided by a synthesizer 19. The selection of corresponding control programs to generate an MR image and the presentation of the generated MR image take place via a terminal 13 which comprises a keyboard 15, a mouse 16 and a monitor 14.

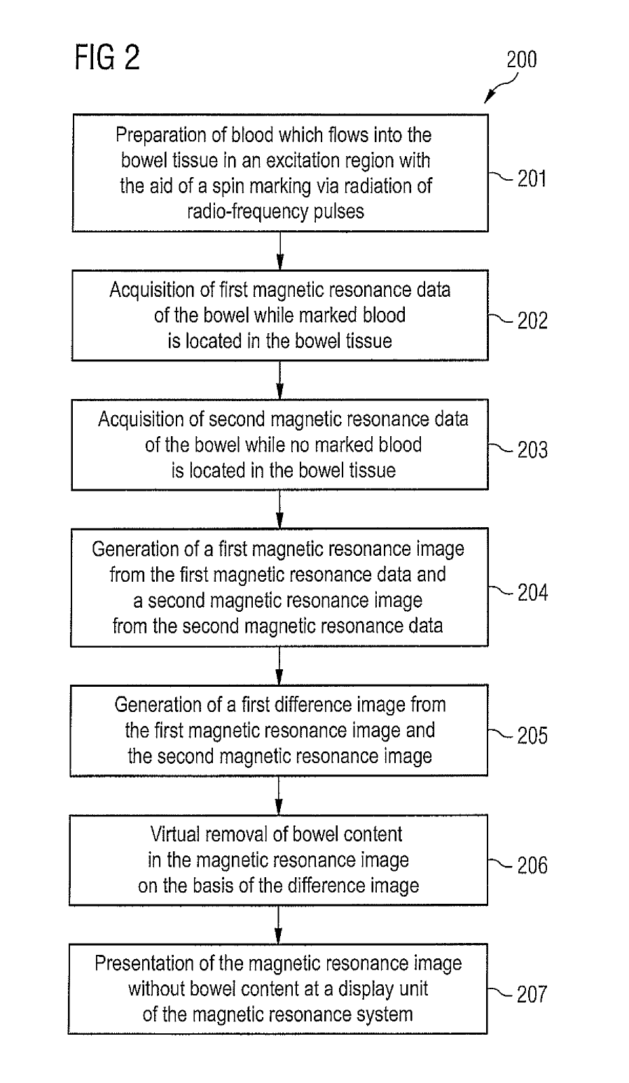

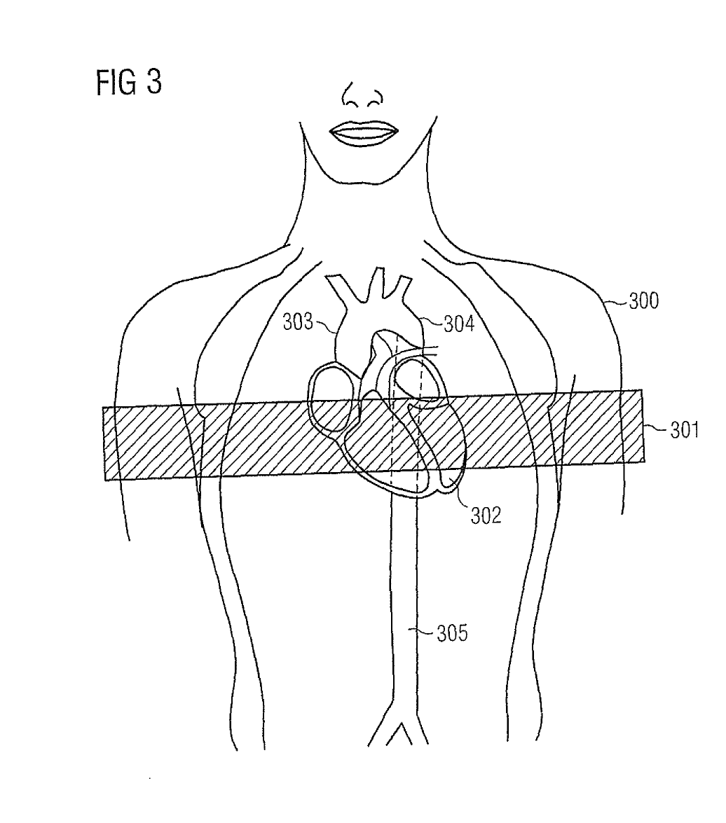

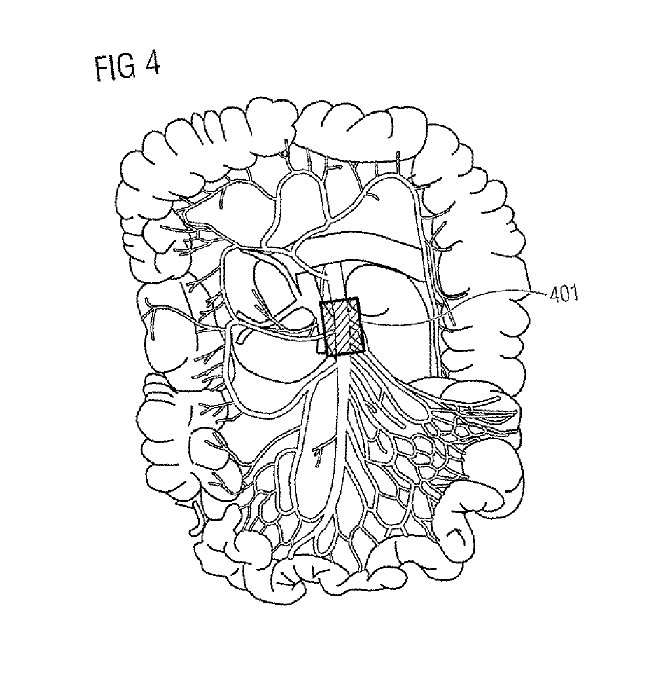

In the following an implementation of a magnetic resonance colonography according to the present invention is described in detail with reference to FIGS. 2-4. For this a workflow diagram 200 that depicts the individual Steps for implementation of the method is shown in FIG. 2. Different excitation regions for a spin marking are shown in FIGS. 3 and 4.

In the method 200, blood which will flow into the bowel of a patient to be examined is prepared in an excitation region in Step 201 with the aid of a spin marking via radiation of radio-frequency pulses. An example of an excitation slice 301 which was selected transversally above the diaphragm of the patient 300 is shown in FIG. 3. With each heartbeat of the heart 302 blood is transported into the ascending aorta 303 and the aortic arch into the descending aorta 304. Vessels for the bowel exit from the lower part 305 of the descending aorta 304, what is known as the abdominal aorta. Due to the marking of the blood in the excitation slice 301 the spins in the excitation slice 301 receive a different magnetic state than those outside of the excitation slice 301, in particular a different magnetization state than spins in the region of the bowel. Due to the blood flow, as described above the marked blood flows into the bowel via the aorta. This alters the magnetization of the bowel tissue. At this time first magnetic resonance data of the bowel are acquired (Step 202), which means that the first magnetic resonance data are acquired while marked blood is located in the bowel tissue. Due to the lasting effect of the magnetic field of the basic field magnet 1, the spin marking of the blood in the bowel tissue is neutralized again in the course of time or, respectively, the marked blood, driven by the cardiac activity, flows out of the bowel tissue. After a predetermined period of time it can therefore be assumed that marked blood is no longer located in the bowel tissue, and second magnetic resonance data of the bowel are acquired at this time (Step 203).

In Step 204 a first magnetic resonance image is generated from the first magnetic resonance data and a second magnetic resonance image is generated from the second magnetic resonance data. A difference image is then generated in Step 205 from the first magnetic resonance image and the second magnetic resonance image, for example in that a difference of the pixel values of the corresponding pixels of the first and second magnetic resonance image is calculated for each pixel of the difference image. The difference image therefore essentially only shows a signal value different than zero in the regions in which marked blood was present at the acquisition of the first magnetic resonance data. Since the bowel wall and the surrounding tissue are generally well-perfused, a precise and reliable differentiation of bowel tissue and the internal space of the bowel (what is known as the gut lumen) is possible with the aid of the difference image. Bowel contents (for example stool residues) thus can be reliably differentiated from bowel tissue. In this way the bowel content can be virtually removed, whereby a previous complete bowel evacuation is not necessary. For example, the bowel content can be removed automatically in the first or second magnetic resonance image (Step 206). Finally, the first or the second magnetic resonance image is shown without bowel content at the terminal 13 of the magnetic resonance system 5.

Intestinal polyps (which can project into the gut lumen, for example) are generally likewise perfused, such that these can be clearly and automatically differentiated from bowel content. Intestinal polyps which project into the gut lumen are therefore clearly recognizable in the first or second magnetic resonance image. The use of the first magnetic resonance image—i.e. of the magnetic resonance image which was acquired while marked blood was located in the bowel tissue—moreover offers an additional advantage: the contrast of perfused tissue is increased by the prepared blood. The perfusion of the polyps can therefore be simply established using the contrast and serve as a measure of the risk of a malignancy of the intestinal polyps.

The excitation slice 301 shown in FIG. 3 can if necessary be expanded upward to the aortic arch insofar as that this is possible in the magnetic resonance system 5. The reliability of the spin marking of the blood can thereby be further improved.

However, a smaller volume can alternatively also be selectively excited. FIG. 4 shows an example of such an excitation volume 401 in which individual branches of the mesenteric artery are excited. One advantage of this excitation is that the vessel relationships can be examined simultaneously, in particular the supply areas of the abdominal aorta above and below the renal vessels, which is significant for a planning of an operation.

In the embodiment described in the preceding, magnetic resonance data were acquired and subtracted with and without selective excitement. However, it is also alternatively possible to acquire magnetic resonance data only with selective excitement of the incoming blood, and to enable a separation of bowel wall and bowel content solely using the increase of the contrast between the two.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for contrast agent-free magnetic resonance colonography, comprising:
automatically preparing blood flowing in bowel tissue in the bowel of a patient located in a magnetic resonance data acquisition unit, by emitting radio frequency pulses in said magnetic resonance data acquisition unit that interact with nuclear spins in said blood, to produce prepared blood;
operating the magnetic resonance data acquisition unit to acquire magnetic resonance data from the patient while the prepared blood is located in the bowel of the patient, and generating a magnetic resonance image of the bowel of the patient from the magnetic resonance data; and
in a processor, automatically differentiating bowel tissue in said image and regions in said image representing bowel content by an increased contrast of said bowel tissue in said image caused by said preparation of said blood exhibited by tissue regions perfused by said prepared blood, compared to bowel content that is not perfused by said prepared blood.

2. A method as claimed in claim 1 comprising:
operating said magnetic resonance data acquisition unit to acquire additional magnetic resonance data from the patient while unprepared blood, that has not been prepared by said radio frequency pulses, is located in said bowel tissue, and generating an additional magnetic resonance image from said additional magnetic resonance data; and
augmenting the automatic differentiation of bowel tissue and bowel content in said magnetic resonance image obtained while said prepared blood was located in the bowel, using said additional magnetic resonance image.

3. A method as claimed in claim 2 comprising using said additional magnetic resonance image to augment said automatic differentiation by comparing said additional magnetic resonance image with the magnetic resonance image obtained while the prepared blood was located in the bowel of the patient, and designating regions that are substantially identical in said additional magnetic resonance image and said magnetic resonance image obtained while said prepared blood was in the bowel, as representing bowel content.

4. A method as claimed in claim 2 comprising augmenting the automatic differentiation using said additional magnetic resonance image by generating a difference image between said additional magnetic resonance image and the magnetic resonance image obtained while the perfused blood was present in the bowel of the patient.

5. A method as claimed in claim 2 comprising acquiring said additional magnetic resonance data with a higher resolution than the magnetic resonance data acquired while the prepared blood was located in the bowel of the patient so that regions associated with the bowel content automatically appear as blank regions in said additional magnetic resonance image.

6. A method as claimed in claim 1 comprising preparing said blood by radiating said radio frequency pulses in a transverse excitation slice of the patient located above the diaphragm of the patient.

7. A method as claimed in claim 6 comprising slice-selecting said excitation slice to extent cranially to the aortic architecture in the patient.

8. A method as claimed in claim 1 comprising preparing said blood flowing into the bowel tissue in a excitation region of the patient that comprises the descending aorta at a level of passage of the descending aorta through the diaphragm of the patient.

9. A method as claimed in claim 1 comprising preparing said blood flowing into the bowel tissue by a selected excitation of individual branches of the mesenteric artery.

10. A method as claimed in claim 1 wherein preparing said blood flowing into the bowel tissue comprises:
a first automatic preparation of said blood flowing into the bowel tissue, in which a sagittal slice along the aorta of the patient is excited by said radio frequency pulses, and in which first magnetic resonance data of the bowel are acquired while the blood prepared by said first automatic preparation is located in the bowel; and
a second automatic preparation of said blood blowing into the bowel tissue, in which a coronal slice along the aorta of the patient is excited by said radio frequency pulses, and in which second magnetic resonance data of the bowel are acquired while the blood prepared by said second automatic preparation is located in the bowel.

11. A magnetic resonance apparatus for contrast agent-free magnetic resonance colonography, said magnetic resonance apparatus comprising:
a magnetic resonance data acquisition unit;
a control unit configured to automatically prepare blood flowing in bowel tissue in the bowel of a patient located in the magnetic resonance data acquisition unit, by operating said magnetic resonance data acquisition unit to emit radio frequency pulses in said magnetic resonance data acquisition unit that interact with nuclear spins in said blood, to produce prepared blood;
said control unit being configured to operate the magnetic resonance data acquisition unit to acquire magnetic resonance data from the patient while the prepared blood is located in the bowel of the patient, and to generate a magnetic resonance image of the bowel of the patient from the magnetic resonance data; and
said control unit being configured to automatically differentiating bowel tissue in said image and regions in said image representing bowel content by an increased contrast of said bowel tissue in said image caused by said preparation of said blood exhibited by tissue regions perfused by said prepared blood, compared to bowel content that is not perfused by said prepared blood.

12. A non-transitory computer-readable storage medium encoded with programming instructions, said storage medium being loaded into a computerized control and evaluation system of a magnetic resonance data apparatus, comprising a magnetic resonance data acquisition unit, said programming instructions causing said computerized control and evaluation system to:
automatically prepare blood flowing in bowel tissue in the bowel of a patient located in the magnetic resonance data acquisition unit, by emitting radio frequency pulses in said magnetic resonance data acquisition unit that interact with nuclear spins in said blood, to produce prepared blood;
operate the magnetic resonance data acquisition unit to acquire magnetic resonance data from the patient while the prepared blood is located in the bowel of the patient, and generating a magnetic resonance image of the bowel of the patient from the magnetic resonance data; and
automatically differentiate bowel tissue in said image and regions in said image representing bowel content by an increased contrast of said bowel tissue in said image caused by said preparation of said blood exhibited by tissue regions perfused by said prepared blood, compared to bowel content that is not perfused by said prepared blood.
